(19) United States
(12) Patent Application Publication
(10) Pub. No.: US 2021/0134172 A1
(43) Pub. Date: May 6, 2021

(54) Computer User Interface Relating to Memory Improvement

(71) Applicant: Time Active LLC, Keller, TX (US)

(72) Inventor: Zachary Zhou, Keller, TX (US)

(73) Assignee: Time Active LLC, Keller, TX (US)

(21) Appl. No.: 16/674,339

(22) Filed: Nov. 5, 2019

(51) Int. Cl. G09B 7/02; G09B 19/00; A61B 5/00; G06Q 50/20

(52) U.S. Cl. CPC G09B 7/02; G06Q 50/20; A61B 5/742; G09B 19/00

(57) ABSTRACT

User interfaces and related method are disclosed for facilitating electronic retrieval-based learning and providing scaffolding and feedback that improve the benefits thereof. Embodiments provide an interface for enabling a number of elements comprising the learning material to be displayed or concealed, thereby enabling a user to fill in the “blanks” that are generated, and revealing the concealed elements in a specific order and at a specific rate, thereby efficiently providing the user with timely feedback that promotes learning. Additionally, the interface provides statistics that a user can use to gain insight into their study performance and enables the user to schedule times to review the learning material based on an estimation of the user's recollection of the learning material at a given time.

### FIELD OF THE INVENTION

The present application relates to computerized user interfaces in the field of electronic learning and memory improvement.

### BACKGROUND OF THE INVENTION

Studies show that retrieval-based learning is more effective than popular studying strategies such as rereading and highlighting, and that retrieval-based learning promotes learning of a variety of types of information. Retrieval-based learning occurs whenever one retrieves previously learned information from memory and retention of that information is later measured. For example, a person can engage in retrieval-based learning by answering fill-in-the-blank questions or using flashcards. Furthermore, research indicates that providing scaffolding (i.e., initially providing retrieval cues to enable successful retrieval of information and then removing those cues) and feedback (i.e., the process of receiving input from the environment based upon the actions or output of a system) increase the benefits of retrieval-based learning.

While it is known that computer systems can assist in retrieval-based learning, the user interfaces presented by prior art learning systems are awkward to use and inefficient. For example, while computerized versions of fill-in-the-blank systems are well known, such systems require the user to manually set up the queries and prompts, and to provide manual input to submit an answer to the prompts, which can be very tedious and inefficient. As for flashcard-based computer interfaces, these prior art systems require a significant amount of time and effort to create, and the flashcard interfaces fail to provide scaffolding or feedback. As a result, studying through prior art user interfaces can be laborious and difficult.

### SUMMARY

Presented herein are a system and method for creating an improved user interface for studying (hereinafter referred to as the “user interface”, the “UI”, or the “study UI”) relating to memory improvement by facilitating retrieval-based learning and providing scaffolding and feedback that improve the benefits thereof. This invention facilitates retrieval-based learning by allowing users to complete fill-in-the-blank questions that the study UI automatically generates from text or an image the user uploads onto a server system, and provides effective feedback by efficiently revealing the answer to the fill-in-the-blank questions. The study UI creates fill-in-the-blank questions by concealing randomly selected portions of the learning material, thereby making them no longer visually intelligible. The study UI can conceal a randomly selected portion in a variety of ways, such as by blurring it, making it 100% transparent, or replacing the content with filler content like “*”. All of these concealment methods, as well as others, can be implemented using known programming techniques.

In one embodiment, the method includes uploading learning material onto a server system, opening the study UI, using the study UI to conceal a percentage of the number of elements comprising the learning material corresponding to a concealment value setting (hereinafter referred to as ‘memorization level setting’), and using the study UI to reveal the concealed elements of the learning material in a specific order and at a specific rate. In another embodiment, the method also includes using the study UI to schedule a date for a review session to review the learning material and receiving an electronic alert when the user's retention has reached an adjustable threshold level.

A user can upload learning material onto the server system by uploading a file or by copying text or an image and pasting it into the server system. This process creates a note, where the note is the learning material uploaded by a user onto the server system. When the user selects a note, the server system displays the learning material via the study UI. A user can use the study UI to initiate various functions. For example, a user can use the study UI to conceal a percentage of the learning material corresponding to a memorization level setting. In so doing, the study UI obscures portions of the learning material so that the portions are no longer visually intelligible, thus creating “blanks” in the learning material that a user can “fill” by retrieving information from his memory.

In one embodiment, the study UI allows a user to use a feature called autoplay to automatically reveal the concealed elements of a note, thus providing feedback to the user at a specific rate, which the user can adjust. The study UI executes autoplay by scanning both visible and concealed elements of the learning material in a specific order and at a specific rate, identifying whether the element is visible or concealed, and revealing the element if the portion is concealed.

In one embodiment, the study UI allows a user to initiate the autoplay function via an input. For example, a user may press a keyboard shortcut to initiate the autoplay function.

Other embodiments of the methods and systems for creating an improved user interface for learning are described herein. This description is meant to fully describe the system and method but not limit their design, function, or application.

### BRIEF DESCRIPTION OF THE DRAWINGS

FIG. 1 is a schematic view of a system capable of generating the described user interfaces.

FIG. 2 is schematic view of a first embodiment user interface.

FIG. 3 is a flow chart showing a first embodiment method for generating the described user interfaces.

FIG. 4 is schematic view of a second embodiment user interface.

FIG. 5 is a schematic view of a third embodiment user interface.

### DETAILED DESCRIPTION

### System 100

The present systems and methods are understood more readily by reference to the following detailed description, examples, drawing, and claims. However, before the present systems and methods are disclosed, it is to be understood that this invention is not limited to the present systems and methods disclosed unless otherwise specified. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial method, process and results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

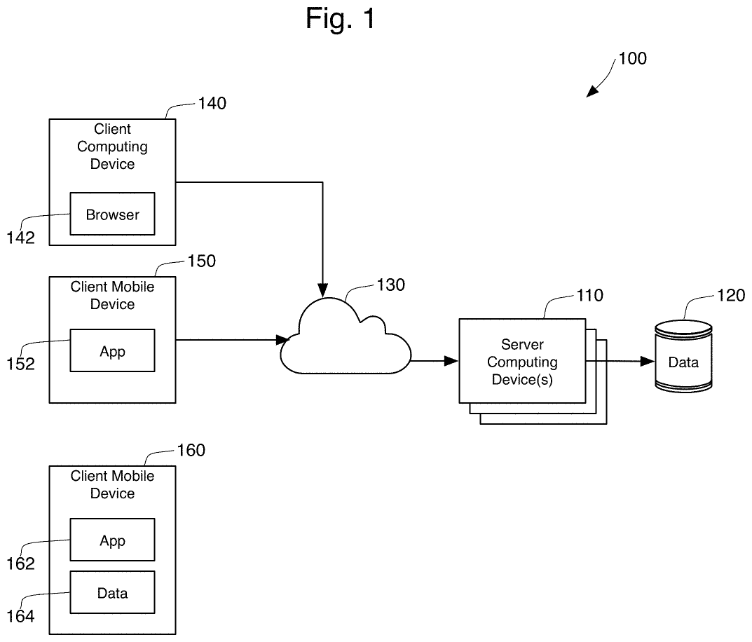

FIG. 1 shows system 100 that provides the user interface of the present invention. In one embodiment, the user interfaces are provided using an online platform, such as a website, that facilitates retrieval-based learning. In this embodiment, one or more server computing devices 110 are responsible for controlling the provided user interfaces and providing the appropriate data 120. The data 120 can be integrated within the one or more server computer devices 110 or can be separately stored such as on an external database. In one embodiment, the database 120 may be managed by its own database management computer system. In other embodiments, the data 120 is stored external to the server computers 110, but the data management is handled by the processors of the server computers 110 directly.

FIG. 1 shows two types of client devices 140, 150 that access the server computer 110 over a network 130. One possible design for the system 100 is having a client computing device 140 operate a web application or browser 142 and communicate with the server computing devices 110 over the web 130. Client 140 communicates with the server 110 over HTTP protocol (which can be secured using SSL). In this embodiment, the client application takes the form of a JavaScript/HTML webpage accessible through browser software 142 operating on the client 140. It is served from a CDN (content delivery network) operated by the server computing devices 110 to provide solid availability and fast load time anywhere in the world. The user interfaces are provided by a fleet of web servers 110. The number of servers 110 is dynamic and reacts (both up and down) to the current traffic amount from the client applications 142 via a load balancer (not shown). It is also known that all incoming traffic at the server computers 110 can flow through a firewall or API gateway (also not shown) that tackles malicious requests such as DDoS attacks or SQL injections. In order to make the system 100 fault-tolerant, web servers 110 are spread over multiple data centers in different regions. If one data center goes down, the traffic is automatically routed to the remaining locations. Application data can be stored in a relational database server cluster 120. This data 120 is replicated using a master-slave scheme, where one server 110 acts as a primary node and other servers 110 are read replicas. This allows the system 100 to remain available as read-only if something happens to the master node instead of just being not responsive. In addition, frequently used data is cached in an in-memory key-value storage, which can be maintained by the computing devices managing data 120.

In one embodiment, the network 130 is a wide area network such as the Internet or a TCP/IP-based Intranet, and the network interfaces on devices 110, 140, 150 include TCP/IP protocol stacks for communicating over the network 130. The network interfaces may connect to the network 130 wirelessly or through a physical wired connection.

In another embodiment, a specially created application or app (other than a standard web browser) operates on a client device, such as the app 152 operating on client mobile device 150. The app 152 then sends data requests to and receives relevant data from the server computing devices 110. In contrast to the browser 142, which simply displays the web page interface created by the web server computing devices 110, the app 152 remains fully responsible for creating the user interface seen by the user. The server computer devices 110 do not format the user interface, but merely provide data from data store 120 for use by the app 152. The app 152 receives this data and incorporates the data into the user interface created by the app 152.

In another embodiment, the system 100 uses an offline application or app, such as an app 162 that is operating on mobile device 160. With this app 162, all data 164 is stored locally on the client device 160. There is no need to access the server computers 110 over network 130, since the app 162 is fully responsible for creating the user interface and all data 164 is stored locally and is directly accessible to the app 162.

The client computing devices 140, 150, 160 and the server computing devices 110 are all similar, in that they all utilize a general-purpose processor and an operating system to run and manage computer programs and data. Non-mobile devices, such as client device 140 and server computing devices 110, will most likely use a processor provided by Intel Corporation (Santa Clara, Calif.) or Advanced Micro Devices (Sunnyvale, Calif.), and will use an operating system provided by Microsoft Corp. (Redmond, Wash.) or Apple, Inc. (Cupertino, Calif.), or an operating system provided under an open source license such as Linux. Mobile devices, such as client devices 150, 160, will use a mobile specific processor such as those provided by Apple, Inc. or that are constructed according to specifications created by Arm Holdings (Cambridge, England).

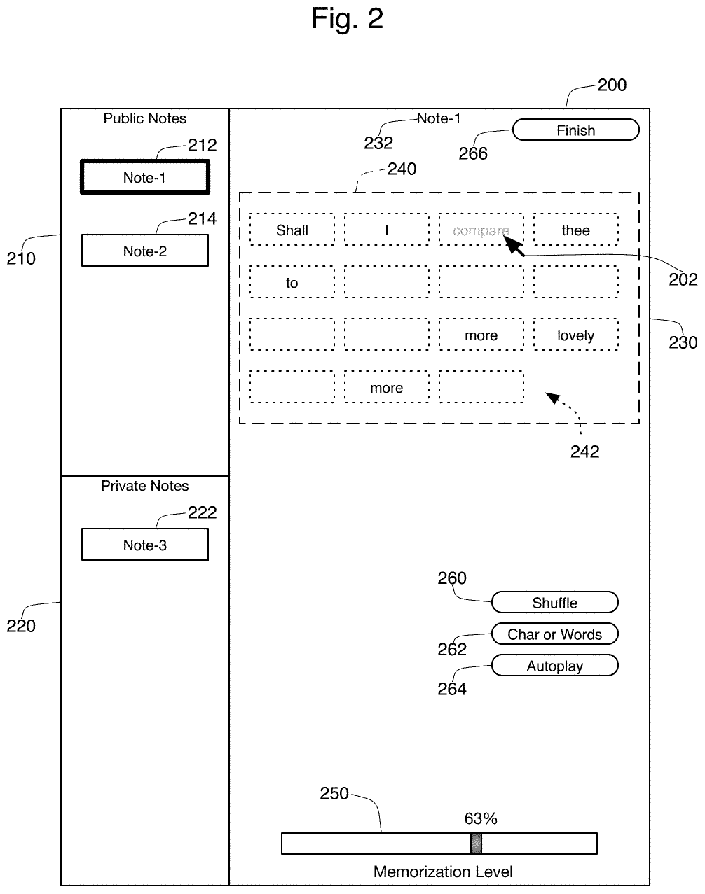

While the system 100 can take a variety of forms (web servers providing web pages to browser 142, servers providing data to custom apps 152, or a local app 162 processing local data 164), in each of these forms the system 100 provides the interfaces described below in connection with FIGS. 2, 4, and 5. While the description below refers to embodiments in which data and notes are uploaded to a server system or service devices 110, the methods and interfaces described apply equally to the local app 162 and data 164 embodiment.

### Study User Interface and Method

This invention is an improved study UI relating to memory improvement, and a method for creating and using that interface. The study UI facilitates retrieval-practice by allowing users to complete fill-in-the-blank prompts that the study UI automatically generates from text or an image provided by the user. The user interface further provides effective feedback by efficiently revealing the hidden material during the study process. The study UI creates a fill-in-the-blank interface by concealing randomly selected portions of the learning material, thereby making them no longer visually intelligible. The study UI can conceal a randomly selected portion in a variety of ways, such as by blurring it, making it 100% transparent, or replacing the content with filler content like “*”. All of these concealment methods, as well as others, can be implemented using known programming techniques.

FIG. 2 shows user interface 200, which is one embodiment of the study UI. This user interface 200 includes a first section 210 for listing stored notes 212, 214 that are available to the public and a second section 220 for listing and selecting private notes such as note 222. A note 212, 214, 222 (which can be either privately or publicly accessible) is a file containing text, images, or both that the user has uploaded onto the system 100 to study using the study UI's features. These notes 212, 214, 222 can be identified by their metadata, such as their title and publication date, which the interface 200 displays. The user may select a note 212, 214, 222 from the first or second sections 210, 220 for further study. Selecting the note opens the note in section 230 of the study UI 200. In FIG. 2, note-1 212 was selected, which is shown by the heavy highlight surrounding note-1 212 and by the fact that the title area 232 of section 230 shows the title for note-1 212. Once the note 212 is selected, a redacted version of the note 212 suitable for study appears in the note presentation area 240.

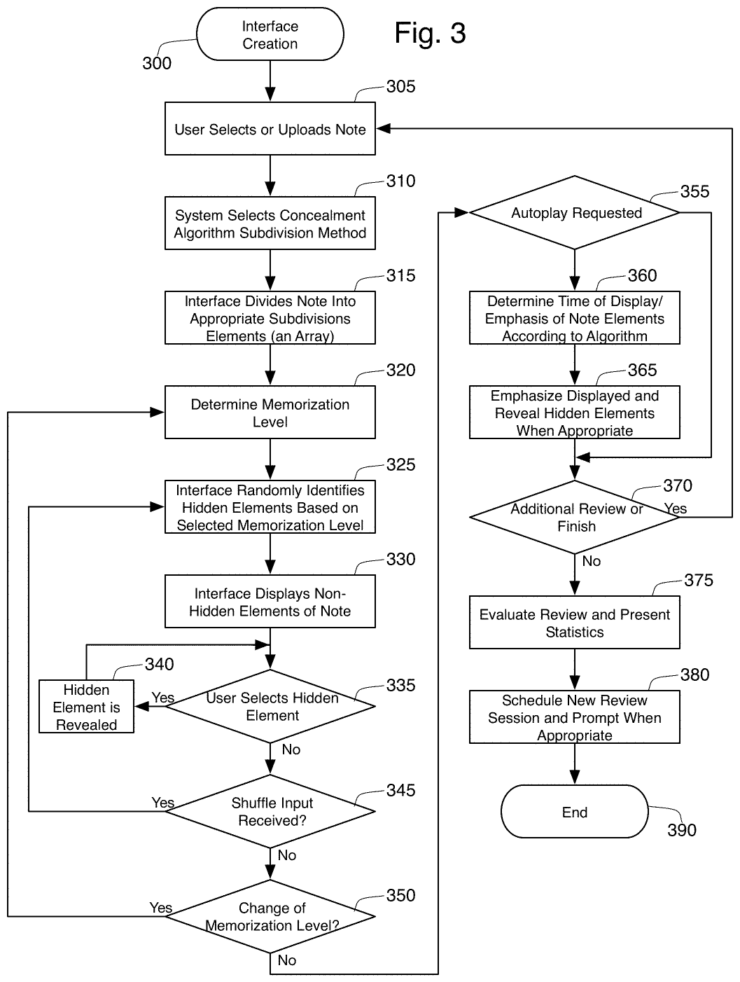

A method 300 for using a study UI is shown in FIG. 3. The method 300 starts with the user selecting or uploading the note at step 305. Sections 210 and 220 display public notes and private notes, respectively, that have been previously identified or created by the user and may be selected at this step 305. In one embodiment, the user can add any note created by other users to their public notes section 210. Selecting a public note created by another user can be considered a subscription to that note (meaning that updates made to the public note by other users will be shown to the user when that note is later selected in section 210), or a personal copy of the public note that is created for the user's own use and modification as they see fit. In one embodiment, the interface 200 uses a browse page (not shown) to let the user search for and select the notes 212, 214, 222. This browse page displays all public notes that all users have created, or all private notes, or both. Regardless of whether the browse page is used or the first and second sections 210, 220 are used, the interface 200 provides a search ability to find a note that the user desires to study.

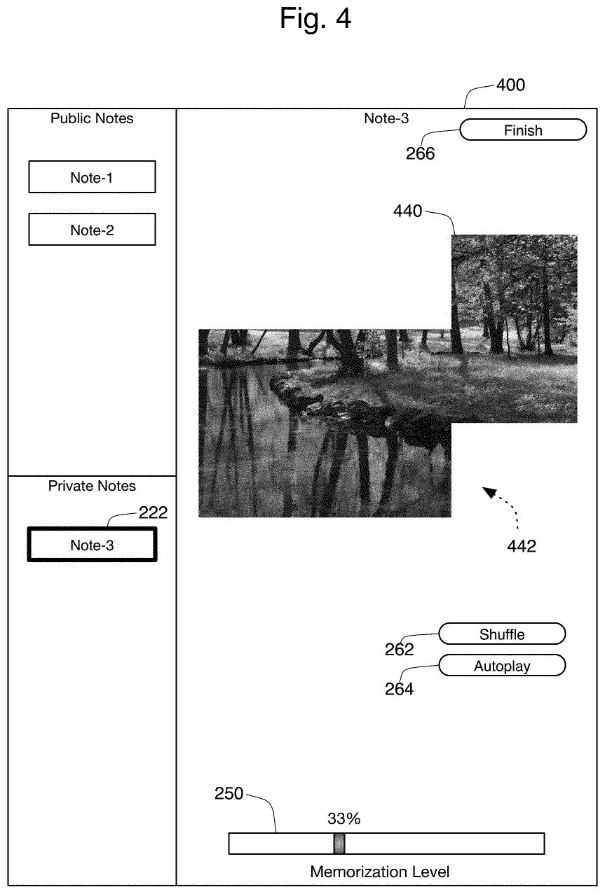

A user can create a new note 212, 214, 222 by uploading text onto the server system 110, such as by copying text and pasting it into the server system (as explained below in connection with FIG. 4, the user can also upload an image). The server system 110 then displays the uploaded material via the study UI. In the stand-alone embodiment used by device 160, the note is stored into the data 164 stored locally on the device rather than on the server system 110.

The study UI 200 conceals a percentage of the learning material of the note by first identifying or selecting the concealment algorithm to be used on the note. For text-based systems, the concealment algorithm may work on characters or words. A character is any single letter, number, space, punctuation mark, or symbol that can be typed on a computer. A word is any number of contiguous non-space characters that is adjacent to a space character on each end (exceptions are made for the first and last word of a note, which cannot have a space character on each end). The selection of the concealment algorithm takes place in step 310 of method 300 and can be made using user interface button 262 in FIG. 2.

The server system 110 or self-contained app 160 next creates an array consisting of all the elements of a note at step 315. The array consists of the individual elements of the notes subdivided according to the concealment algorithm selected at step 310. The array will be sorted appropriately for the material. According to convention in the United States, the chronological order of a series of characters is from left to right and top to bottom; the order of a series of words is from left to right and top to bottom; and the order of a series of portions of an image is from left to right and top to bottom. However, the order could easily be changed if necessary, for example, by applying a mathematical equation to reverse the sorting of the elements.

While method 300 is described herein using the concept of an “array” of elements, it is not necessary that an actual “array” data construct be used when programming systems to perform this step. As is well known in the industry, a variety of data constructs could be used instead of an actual array to subdivide the note into an ordered set of subcomponents.

At step 320, the memorization level for the interface 200 is determined. On the study UI 200 of FIG. 2, the memorization level setting can be adjusted by a user through the memorization level slider 250 at the bottom of the study UI 200. By adjusting the memorization level setting 250, the user can control the percentage of learning material that is concealed during the study session. For example, if the learning material is text, and the user selects a memorization level setting of 63%, then approximately 63% of the text will be concealed.

At step 325, the system 100 randomly conceals selected elements of the array created at step 315, while at step 330 the interface displays the non-concealed elements. One method of determining which elements of the array are to be concealed is by using a numbered array and a randomization function. In this method, the elements of an array are numerically identified by their position in the array. The system 100 then selects a random element by executing a randomization function that outputs a randomly generated number that corresponds with the number of an element in the array. The study UI 200 then conceals that element within the note presentation area 240. The system 100 continues to conceal random elements that have not already been selected in this manner until the number of elements in the array that have been concealed divided by the total number of elements in the array is as close as mathematically possible to the memorization level setting 250 the user selected. It is important to note that the total number of outputs in the set of numbers that can be generated by the randomization function needs to be the same size as the array. Notwithstanding that condition, it is possible to design the study UI 200 so that it conceals the elements in an alternative manner that behaves in substantially the same way as the one disclosed here. It is also important to note that, as indicated above, it may be impossible for the study UI 200 to exactly conceal a specific percentage of the learning material because the study UI 200 is concealing a fraction of the learning material, where the numerator and denominator of that fraction is an integer. In those cases, the study UI 200 conceals a percentage of the learning material as close as mathematically possible to the memorization level setting that the user selected.

As shown in FIG. 2, the note presentation area 240 contains a plurality of words, which indicates that the concealment algorithm subdivision method selected by step 310 was word-by-word. Some of these words in area 240 are visible, while some of the words are not visible. A space or blank is generally presented in the presentation area 240 to indicate to the user that a word from the selected note 212 is missing. While FIG. 2 shows that the concealed words are made invisible or transparent, other concealment methods are possible, such as blurring or obscuring the concealed portions of the note 212.

After step 330, the user can then practice filling in the blanks or spaces in the presentation area by recalling the information from his or her memory. In one embodiment, the study UI allows the user to reveal a concealed element by hovering a mouse over the element (step 335), thereby receiving feedback on a fill-in-the-blank question (at step 340). In FIG. 2, this is shown by mouse pointer 202 hovering over the word “compare” in presentation area 240. The word “compare” is shown in gray in FIG. 2, to indicate that the word is appearing within its box only after the cursor 202 has hovered over it (step 340). In one embodiment, the revelation of the word (such as “compare”) remains only as long as the cursor 202 hovers over the blank or space containing the word. Thus, the user can only reveal one word at a time. This type of selective revelations can greatly aid a user in evaluating his or her memory performance when using the study UI 200.

The study UI 200 includes a button 260 or other interface element that allows the user to request a “shuffling” of the concealed elements in presentation area 240. Pressing the button 260 for the shuffle feature (at step 345) causes the method 300 to re-execute the process of concealing randomly selected portions of the learning material at step 325. The memorization level setting 250 can remain unchanged, but the presentation area 240 will show a new mix of revealed and concealed elements of the selected note 212.

A user can also change how the study UI 200 conceals the content of a note to suit the type of content, thereby facilitating memorization and optimizing learning. For example, if a user wants to study the spelling of a text, the user can change the concealment algorithm setting on the study UI 200 by pressing interface element 262 so that the study UI 200 conceals random characters of a text. This allows the user to practice retrieving from his memory concealed characters. Although not shown in FIG. 3, the selection of button 262 would cause method 300 to return to step 310 to select a new concealment method.

In addition to subdividing text by characters or words, it is also possible to utilize subdivision methods that divide the texts into phrases, sentences, or paragraphs of a text, or even by lines in a play. For example, if a user is memorizing a long passage of text, the subdivision method may subdivide the text into multiple sentences, and then hide a percentage of the sentences based on the selected memorization level 250. This allows the user to practice retrieving from his or her memory the entirety of a concealed sentence.

When the user wishes to change the memorization level for a particular study session, the user can alter the memorization level slider value 250 by an appropriate amount. For example, if the user is satisfied with his or her performance at the current memorization level, the user would increase the memorization level at step 350. The method 300 would then conceal more elements at steps 320-330, thereby providing scaffolding enhancing the benefits of retrieval-based learning.

FIG. 4 shows a version of the study UI 400 in which a user selected a note 222 that consists of an image rather than textual material. The creation of an image-based note 222 is performed in the same manner as text-based note 212, with the user simply uploading an image onto the server system 110 or into the local data storage 164.

By adjusting the memorization level setting 250 on this study UI 400, the user can conceal a percentage of the image 440 that corresponds to the setting the user selected. The study UI 400 conceals portions of the image 440 by dividing the image into a grid having columns of equal width and rows of equal height. In one embodiment, the user can select the number of columns and rows by adjusting an image parameter setting associated with the note 222. In other embodiments, the number of columns and rows is automatically selected based on typical user preferences, such as a 3×3 grid, a 4×5 grid, or a 6×8 grid. The creation of the grid is accomplished as step 315 in method 300. The system 100 then conceals a number of portions of the grid until the area of the image that is concealed divided by the total area of the image is approximately equal to the memorization level setting the user selected. This occurs at steps 320-330, as described above. For example, if a user adjusts the image parameter setting to have the study UI 400 divide the width of an image into 3 sections and the height of the image into 3 sections, then the system will create a 3×3 grid with 9 rectangular portions that the study UI 400 can conceal. These portions of the image will then be randomly selected and concealed so that the percentage of the image that is concealed is as close as mathematically possible to the memorization level setting that the user selected.

There is only one concealment algorithm setting for images (an image-based grid), so the user interface 400 does not include button 262 to select a concealment algorithm when displaying an image. When the user studies an image-based note, the study UI 400 conceals random portions of an image, and the user practices retrieving from his memory concealed portions of an image. Interface 400 still includes the ability of a user to select a concealed portion for temporary revelation using step 335, 340, as described above.

### Autoplay

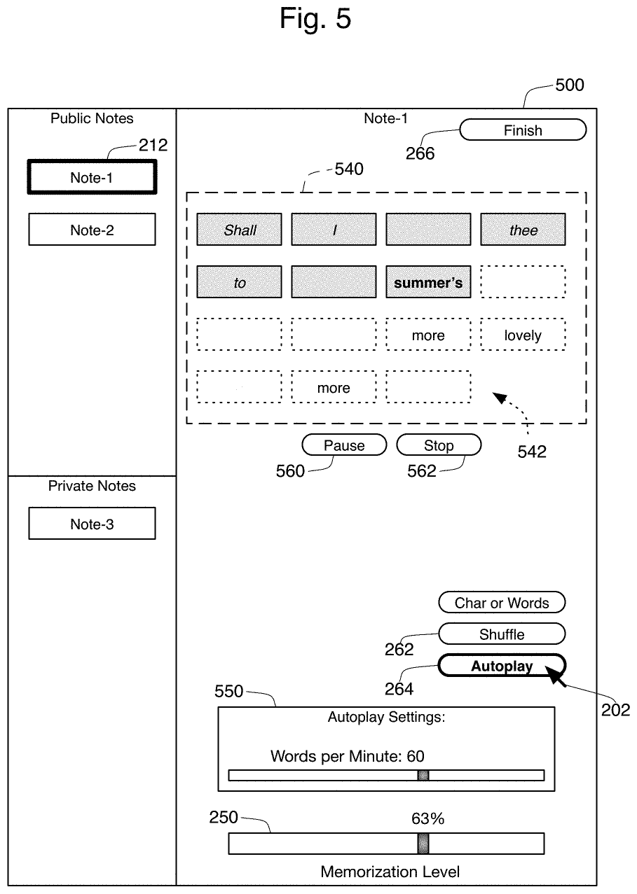

Buttons 264 shown in interface 200 and interface 400 allow the user to perform a function known as autoplay. The selection of the autoplay function is shown as step 355 in method 300. Autoplay allows a user to have all the concealed elements consecutively revealed at a specific rate, and is shown in connection with interface 500 shown in FIG. 5. The autoplay function is available only after method 300 first performs the steps necessary to present a partially concealed version of the selected note. In particular, the method 300 must first identify whether the concealment algorithm setting is set to conceal characters, words, sentences, paragraphs, portions of an image, etc. (at step 310). The system 100 next creates an ordered array consisting of all the elements of a note at step 315. The system 100 then determines which elements in the array to conceal based on the memorization level and a randomization function, and then displays the non-hidden elements (steps 320-330).

The autoplay feature is designed to automatically pass through the entire note to challenge the user to remember the concealed elements before autoplay reveals that element. In order to function, autoplay must determine how much time to spend on each of the note elements during the autoplay function (step 360). There are various possible algorithms for determining the amount of time autoplay spends per element. One autoplay algorithm can have the study UI spend the same amount of time per element. This algorithm simply requires a determination of speed in order to function. For example, if an array includes the word elements ‘Shall’, ‘I’, ‘compare’, and ‘thee’ and the autoplay speed is 60 words per minute, then the study UI shall spend 1 second per word element before the server system proceeds to the next word element.

Another autoplay algorithm for words can have the study UI spend a variable amount of time per word element and have that amount of time change in a linear manner to the number of characters in a word element. In this alternative embodiment, it is necessary to establish a standard number of characters in a word element so that the study UI 500 spends the amount of time prescribed by the autoplay speed setting on a word element with a character length equal to the standard number of characters before proceeding to the next word element. Moreover, it is necessary to establish the slope of the liner relationship that defines how the time the study UI 500 spends per word element varies with the number of characters in the word element. For example, if an array includes the word elements ‘Shall’, ‘I’, ‘compare’, and ‘thee’, the autoplay speed setting is 60 words per minute, and the standard number of characters in a word element is made to be 5, then the study UI 500 shall spend 1 second per word element that is 5 characters long, less than 1 second for word elements having fewer characters than 5 and more than 1 second for word elements having more characters than 5. Accordingly, for the array in the above example, the study UI 500 shall spend 1 second on the word element ‘Shall’, less than 1 second on the word elements ‘I’ and ‘thee’, and more than 1 second on the word element ‘compare.’ This algorithm could also be used in sentences, with a given amount of time spent on a sentence with an average number of words, a shorter period of time spent on shorter sentences, and a longer period of time on longer sentences.

If the slope of the linear relationship that defines how the time the study UI spends per word element varies with the number of characters in the word element is made to be 0.2 seconds per character, then the study UI shall only spend 0.2 seconds on ‘I’ (1 second−0.2 seconds*4 characters=0.2 seconds), 1.4 seconds on ‘compare’, etc. Furthermore, additional rules can be implemented so that there is a minimum and maximum amount of time the study UI shall spend on a word element, regardless of the number of characters in it. It is important to note that establishing a standard character length is arbitrary. It is also important to note that it is possible to define other relationships, both linear and nonlinear, between the amount of time autoplay spends per word and the length of the word that behave in substantially the same way as the one disclosed in this description.

At step 365, the study UI 500 then presents the displayed note in presentation area 540 according to the autoplay algorithm. This display starts with the first array element 542 and emphasizes or displays the element for the amount of time prescribed by the autoplay speed setting algorithm. If the element 542 is concealed, the study UI 500 reveals it for the amount of time prescribed by the autoplay speed setting algorithm, and the interface 500 then proceeds to the next element in the array. In one embodiment, if the element 542 being emphasized according to the algorithm is already revealed, then the study UI emphasizes the visual appearance of the element—for example, by bolding or italicizing it—thereby allowing the user to more easily track autoplay's position in the learning material. In other embodiments, if the element 542 is not being concealed, the study UI 500 makes no change to the appearance of the element 542 and simply delays for the amount of time prescribed by the autoplay speed setting, and then proceeds to the next element 542 in the note 212. In FIG. 5, the elements 542 already displayed or emphasized by the autoplay algorithm are surrounded by a shaded gray background or box.

The element 542 currently being presented is the word “summer's.” The element was previously hidden (see FIG. 2) and is currently being displayed by the autoplay algorithm (hence it is presented in bold and italics). After the time period determined for this element 542 has elapsed, the box containing the word will go blank (rehidden), and the next box will be revealed (which will be the word “day” according to Shakespeare's Sonnet 18).

In one embodiment, a user can adjust the autoplay speed setting by adjusting a slider 550 on the study UI 500. In FIG. 5, the value of this slider 550 corresponds to a specific rate in words per minute although other rates (such as seconds per word) could also be used. The rate will be specified in the same units at the subdivision method (e.g., seconds per character or seconds per sentence). The interface 550 also provides a pause/resume button 560 and a stop/start button 562. The pause/resume button 560 lets the user temporarily interrupt an autoplay of the selected note 212, while the stop/start button 562 ends the autoplay completely and then restarts the session at the beginning of the note 212.

In one embodiment, the system 100 collects data during each study session performed by a user. The system 100 then records, inter alia, the user's usage of the study UI's key functions as a function of time. These key functions primarily consist of the memorization level slider 250 and autoplay performances 264 and rates 550. A user that has better memorized the note 212 will be able to perform autoplay multiple times at high memorization levels 250 and high rates 550. The user can choose to perform autoplay again by pressing autoplay button 264. Before doing so, the user can alter the memorization level 250 and/or the autoplay settings 550. The method would then start again at step 360.

At step 370, the user can elect to study a different note 212, 214, 222, or to find or create a new note, in which case the method would return to step 305. If the user is finished using the study UI 500, a user can click a finish button 266 (which is also shown on interfaces 200 and 400). Upon clicking the finish button 266, the study UI 500 will evaluate the user's review session and then display statistics pertaining to the user's performance using the study UI 500 (step 375). These statistics include the length of the study session for each note, the number of words in the notes, the number of words that the user memorized, the average words memorize per minute (WMPM), autoplay rates (such as words per minute or WPM), the minimum non-zero memorization level selected, the maximum memorization level selected, and the session number. The session length is defined as the amount of time that elapsed between the time the user selected their first note 212, 214, 222 for study and the time the user clicked the finish button 266. The total number of words is defined as the number of words in the note being reviewed. The number of words memorized is defined as the total number of words in the reviewed note multiplied by the maximum memorization level selected 250 for that note. The average WMPM is defined as the total number of words memorized divided by the session length. The minimum non-zero and maximum memorization levels are the minimum non-zero value of the memorization level slider and the maximum value of the memorization level slider respectively.

It is important to note the user is not providing any input for the study UI 500 to evaluate as “correct” or “incorrect” answers. This means that the user needs to evaluate his own performance when using the system 100.

In one embodiment, the user can take advantage of the spacing effect by scheduling review sessions on the study UI (step 380). The system 100 allows a user to schedule a review session by entering a maximum memorization level for a note, the minimum memorization level for a note, and a review memorization level for a note. The system 100 then calculates the rate at which a user's theoretical memorization level (i.e., the highest memorization level the user can theoretically use for retrieval-based learning for that note following the study session) decays over time, and the study UI sends the user a notification via the user's preferred method (e.g., email or text) at the time when the user's memorization level is predicted to reach the review memorization level selected by the user. For example, assuming that a user's memorization level decays linearly over time and that the slope of this line changes with each review of the same note (i.e., the amount of time between review sessions for a user's memorization level to decay to a review memorization level increases or decreases), the system 100 can calculate an estimation of when the user needs to review that note, and the system 100 will send the user a notification via the user's preferred method. It is important to note that scheduling review sessions based on memorization level values is useful because doing so uses individual performance statistics to determine when a user needs to review, which can result in higher studying efficiency for that individual than if general performance statistics were used. Also, allowing a user to select the memorization levels 250 that are used to schedule review sessions allows the user to control the frequency of the reviews and the strength of his memory of the learning material at any given time, since memorization level 250 is correlated with retention of the learning material.

The preferred definitions and formula for scheduling a review session are as follows:
1) The maximum memorization level is the largest memorization level executed in the immediately prior session for a note.
2) The minimum memorization level is the smallest memorization level executed in the current session for a note.
3) The review memorization level is the threshold memorization level selected by the user; when the maximum memorization level decays and reaches the review memorization level, the study UI sends a notification to the user.
4) The time elapsed is the time between the start of the current session for a note and the end of the immediately prior session for the same note.
5) Rate of memory loss is (maximum memorization level-minimum memorization level)/time elapsed.
6) The number of hours later a review session should be scheduled is (maximum memorization level for the current session-review memorization level)/rate of memory loss.

It is important to note that if a user reviews a note immediately after scheduling a review session and has a minimum memorization level equal to or greater than his maximum memorization level for the immediately prior session, the minimum memorization level needs to be replaced with a value less than the maximum memorization level to calculate the rate of memory loss. It is also important to note that if the final memorization level is equal to or less than the critical point, the review session date should be scheduled some arbitrary non-negative amount of time in the future (e.g., 1 or 5 seconds later) to avoid an irrational result—for example, a review session scheduled a negative amount of time later.

In one embodiment, the scheduling review session step 380 operates by sending the user a notification via the user's preferred method at the appropriate time. The advantage of the former method, though, is that because it uses personal user data, it is likely to be more accurate and precise. Regardless of how the user schedules a review session, the invention can send notifications to tell the user when the time for the scheduled review session has arrived.

In a further embodiment, a user can use keyboard shortcuts to have the invention perform various actions. These actions include, but are not limited to, increasing the memorization level by increasing the value of the memorization level slider 250, decreasing the memorization level by decreasing the value of the memorization level slider 250, starting, pausing, and resetting autoplay 264, and changing the concealment algorithm 262.

The many features and advantages of the invention are apparent from the above description. Numerous modifications and variations will readily occur to those skilled in the art. Since such modifications are possible, the invention is not to be limited to the exact construction and operation illustrated and described. Rather, the present invention should be limited only by the following claims.

What is claimed is:

1. A method of generating a user interface for an electronic device comprising:
a) receiving through the user interface a selection of a note for review;
b) dividing the selected note into a total number of elements for review;
c) receiving through the user interface a selection of a memorization level setting convertible to a percentage of elements to be concealed;
d) determining a number of concealed elements by multiplying the percentage of elements to be concealed against the total number of elements;
e) selecting the number of concealed elements as concealed elements;
f) presenting the note through the user interface while concealing the concealed elements of the note, thereby producing a blank area in the presentation of the note for each concealed element;
g) determining a time period for each element for autoplay; and
h) starting at the beginning of the note, emphasizing each element of the note for its time period, wherein concealed elements are revealed during their time period.

2. The method of claim 1, wherein the time period for each element is the same for all elements of the note, thereby creating an unchanging rate for emphasis.

3. The method of claim 1, wherein the time period for each element differs in proportion to a length of the element.

4. The method of claim 1, wherein each element that is not concealed is altered in appearance during its time period and reverts to its original appearance after its time period has passed.

5. The method of claim 1, wherein each concealed element that is revealed remains revealed during the emphasizing step.

6. The method of claim 1, wherein elements that have not been emphasized are displayed differently than elements that have been emphasized so that the progression through the note during the emphasizing step is visually clear.

7. The method of claim 6, wherein elements that have not been emphasized have a shaded background.

8. The method of claim 1, further comprising the step of scheduling a review session based on a negative linear rate of change over time in the selected memorization level.

9. The method of claim 8, further comprising the step of sending the user an electronic alert notifying the user to review the learning material at the scheduled time.

10. The method of claim 1, further comprising the step of scheduling a review session based on an exponential decay rate of change over time in the selected memorization level.

11. The method of claim 10, further comprising the step of sending the user an electronic alert notifying the user to review the learning material at the scheduled time.

12. The method of claim 1, further comprising receiving through the user interface a new memorization level setting convertible to a new percentage of elements to be concealed, further wherein steps d) through steps h) are performed using the new percentage of elements to be concealed.

13. The method of claim 1, further comprising receiving through the user interface a rate selection, further wherein the time period for each element for autoplay is determined based on the rate selection.

14. A method of generating a user interface for an electronic device comprising:
a) receiving through the user interface a selection of a note for review;
b) dividing the selected note into a total number of elements for review;
c) selecting a number of concealed elements as concealed elements;
d) presenting the note through the user interface while concealing the concealed elements of the note, thereby producing a blank area in the presentation of the note for each concealed element;
e) determining a time period for each element for autoplay; and
f) starting at the beginning of the note, emphasizing each element of the note for its time period, wherein concealed elements are revealed during their time period.

15. The method of claim 14, wherein the time period for each element is the same for all elements of the note, thereby creating an unchanging rate for emphasis.

16. The method of claim 14, wherein each element that is not concealed is altered in appearance during its time period and reverts to its original appearance after its time period has passed.

17. The method of claim 14, wherein elements that have not been emphasized are displayed differently than elements that have been emphasized so that the progression through the note during the emphasizing step is visually clear.

18. The method of claim 14, further comprising receiving through the user interface a rate selection, further wherein the time period for each element for autoplay is determined based on the rate selection.
